(19) United States
(12) Patent Application Publication
(10) Pub. No.: US 2012/0184850 A1
(43) Pub. Date: Jul. 19, 2012

(54) Imaging and directed phototherapy catheter

(76) Inventors: Antonio Gutierrez, Durham, NC (US); Gregory A. Imbrie, Newton, MA (US)

(21) Appl. No.: 12/930,747

(22) Filed: Jan. 14, 2011

(51) Int. Cl. A61B 8/00; A61N 5/00

(52) U.S. Cl. 600/439

(57) ABSTRACT

Disclosed are systems, methods and devices for imaging and phototherapy. In one embodiment, a catheter system includes a light emitter and an ultrasound imaging system coupled to the light emitter. The ultrasound imaging system transmits an image to a display device, and the light emitter emits light energy directed at a target location.

### CROSS-REFERENCE TO RELATED APPLICATIONS

This application claims the benefit of U.S. Provisional Patent Application No. 61\284,966, entitled “Percutaneous Atheroma Stabilization System (PASS)” filed on Jan. 14, 2010, the entire disclosure of which is hereby incorporated by reference as if set forth herein in its entirety.

### BACKGROUND

Coronary artery atherosclerosis is a leading cause of death today, and treatment to date has relied on stenting of highly stenosed vessels coupled with aggressive risk factor modification. In sudden coronary death and acute myocardial infarction, lesions resembling plaque rupture (thin-cap fibroatheroma, vulnerable plaque) are reported in other arterial sites remote from culprit lesions. Chronic inflammation derived from macrophage infiltration, foam cell formation, a robust necrotic core, fibrous cap, and degradation of collagen is the pathway of asymptomatic to symptomatic lethal atherosclerotic disease. The aforementioned atherosclerotic lesions are considered to be higher risk for rupture and are believed to cause 10-15% incidence of repeat myocardial infarctions in patients with previous hear attacks. Treatment of smaller, asymptomatic plaque and therapeutic targeting of inflammatory cells in said plaque is a significant goal in treating heart disease.

### SUMMARY

The systems, devices and methods described herein include a catheter with both imaging and directed phototherapy systems. In particular, the systems include visible light therapy coupled with pre-sensitizing chemical agents as a means to target, for example, inflammatory cells. In one application, the system targets inflammatory cells in asymptomatic atherosclerotic plaque thus halting the atherosclerotic plaque's natural evolution into symptomatic vulnerable rupture prone lesions. The catheter system incorporates an imaging modality that can operate independently of the one or more light emitters. Additionally, the light energy output from the light emitters can be aimed in multiple directions and angles, and the intensity of the light energy may be adjustable.

The systems and methods described herein relate to medical (or veterinary) treatment using photodynamic therapy. According to one aspect of the invention, a catheter system for imaging is provided. The catheter system includes a light emitter and an ultrasound imaging system coupled to the light emitter. The ultrasound imaging system transmits an image to a display device, and the light emitter emits light energy directed at a target location. In one embodiment, the ultrasound imaging system is a distinct component from the light emitter, and the ultrasound imaging system can be operated independently from the light emitter. In some embodiments, the light energy from a selected light emitter may be aimed in different directions, at various angles from the catheter system. The light energy may be photodynamic therapy. The image transmitted from the ultrasound imaging system may be used to identify a target location for photodynamic therapy.

In some embodiments, the system may be a catheter system for imaging and photodynamic therapy in a human or animal patient. The patient may be given a photo-sensitizing agent in conjunction with the therapy.

### BRIEF DESCRIPTION OF THE FIGURES

The foregoing and other objects and advantages of the invention will be appreciated more fully from the following further description thereof, with reference to the accompanying drawings, wherein:

FIG. 1 depicts a side view of a catheter system including three light emitters and an ultrasound imaging system targeting an atherosclerotic plaque in an arterial wall, according to an illustrative embodiment of the invention;

FIG. 2 depicts a partial cut-away view of a catheter with four light emitters and an ultrasound probe, according to an illustrative embodiment of the invention; and

FIG. 3 depicts a side view of a catheter, including an ultrasound imaging system and three light emitters emitting light energy at various angles, according to an illustrative embodiment of the invention.

### DETAILED DESCRIPTION

The systems and methods described herein relate to medical devices for targeted photodynamic therapy from a catheter. For example, the systems and methods may be used in atherosclerotic coronary arteries, carotid arteries, the aorta. In other examples, the systems and methods may be used in other vesicles such as the intestines, urethra, trachea, esophagus, stomach, and cavities created by laparoscopic procedures. The medical device includes a system to enable accurate identification and targeting of a target (e.g., plaque, tissue, lesion), and another system to treat the target. In one embodiment, the systems and methods described herein relate to a cardiac catheter device that utilizes intravascular ultrasound (IVUS) coupled with visible light emittance to enable accurately targeted photodynamic therapy of atherosclerotic coronary arteries.

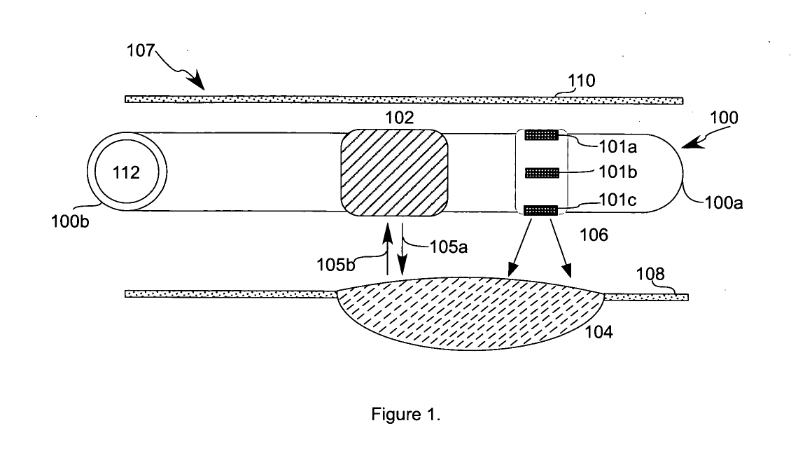

FIG. 1 depicts a side view of a catheter system 100 inside an artery 107, according to an illustrative embodiment of the invention. The catheter system 100 has a distal end 100a and a proximal end 100b, and it may be used percutaneously, with the proximal end 100b outside the patient and the distal end 100a inserted into the patient. The catheter system 100 includes light emitters 101a, 101b, 101c, an ultrasound imaging system 102, and a lumen tube 112. The lumen tube 112 is flexible and open, such that a guide wire could be inserted therein. The catheter system 100 is a flexible elongated device. Artery 107 includes arterial walls 108, 110, and atherosclerotic plaque 104.

In one embodiment, the properties of individual light emitters 101a-101c and of the combination of the array of light emitters 101a-101c allow light energy 106 to be directed to a portion of the surrounding arterial wall 108. The light energy 106 is targeted directly at the atherosclerotic plaque 104, and does not contact the untargeted arterial wall 110. The light emitters 101a-101c can be oriented to direct the light energy 106 at angles distal or proximal to the location of the light emitters 101a-101c. The direction of the light energy 106 can be altered by a user while the catheter system 100 is inside the artery 107.

The ultrasound imaging system 102 may be used to locate a target for the light energy 106. The ultrasound imaging system 102 may be operated independently of the light emitters 101a-101c. In some embodiments, the ultrasound imaging system 102 is connected to a display device which displays ultrasound images created by the ultrasound imaging system 102. The ultrasound imaging system 102 transmits dynamic information, and the display device may display dynamic images which represent the tissue currently targeted by the ultrasound rays 105a. The display device may be a computer or television screen, and is located outside the area where the catheter system 100 is inserted (for example, outside a patient).

In FIG. 1, the ultrasound imaging system 102 emits ultrasound rays 105a which reflect back to the ultrasound imaging system 102. In one example, using the reflected ultrasound rays 105b, the ultrasound imaging system 102 creates an image of the local tissue, which may be displayed on a display device. In another example, the ultrasound imaging system 102 sends data received about the reflected rays 105b to a computer which creates an image of the local tissue. In another example, using the reflected ultrasound rays 105b, the ultrasound imaging system 102 interprets the reflected rays 105b and identifies target tissue. A user may view an image created by the ultrasound imaging system 102 and identify the atherosclerotic plaque 104. In one example, the ultrasound imaging device 102 and the light emitters 101a-101c may be used in conjunction with a photosensitizing agent. The combination of the ultrasound imaging device 102, the light emitters 101a-101c and a photosensitizing agent may be selected to cause apoptosis and cell death of inflammatory cells.

Photosensitizing agents may include drugs classified as porphyrins, chlorophylls and dyes, and may include but are not limited to tetracycline, aminolevulinic acid (ALA), porfimer sodium, verteporfin, temoporfin, methyl aminolevulinate, talaporfin, motexafin lutetium, 2-(1-Hexyloxyethyl)-2-devinyl pyropheophorbide-a (HPPH), xanthotoxin.

In one example, the catheter system 100 is an endovascular catheter system, with the light emitters 101a-101c coupled with the ultrasound imaging system 102 for the individualized and targeted treatment of inflammatory cells of atherosclerotic arterial plaque 104. The light emitters 101a-101c and ultrasound imaging system 102 may be in series, as shown in FIG. 1. In another example, the ultrasound imaging system 102 may be located at the distal end of the catheter system 100, with the light emitters 101a-101c located more proximally. In other examples, the light emitters 101a-101c and the ultrasound imaging system 102 are at the same probe site. In some embodiments, the catheter system 100 may contain any number of independently controlled light emitters 101a-101c. For example, the catheter system 100 may include about one, about two, about three, about four, about five, about six, about seven, about eight, about nine, about ten, about twelve, about fifteen, about eighteen, about 20, about 25, about 30, about 35, about 40, about 45, about 50, about 75, about 100, about 125, about 150, about 200, or more than 200 light emitters 101a-101c.

In one example, the catheter system 100 is inserted in a peripheral artery and subsequently moved into a coronary artery, such as artery 107, for imaging of atherosclerotic plaque, such as atherosclerotic plaque 104. The catheter system 100 uses the ultrasound imaging system 102 to identify the atherosclerotic plaque 104 and then uses the light emitters 101a-101c to target light therapy via light energy 106 to the plaque 104.

In one example, the catheter system 100 is used in a patient's arteries. Through intravascular use of the ultrasound imaging system 102, a user may identify vessel stenosis, and may be able to characterize the plaque structure and stability. In some embodiments, the catheter system 100 is combined with other independent imaging sources, such as prior images and other real-time images, for diagnostic purposes. In one embodiment, the catheter system 100 uses intravascular ultrasound to locate and calculate plaque dimensions—location, depth, and length—throughout the coronary artery system. The plaque dimensions are diagnostic information that can be used to calculate the ideal light intensity, dose duration, and light source angle for each plaque. This may be determined by a user or operator, or by a control unit. This enables the catheter to apply a specific plaque stabilizing treatment dose of light energy for each unique atherosclerotic plaque. Treatment of the plaque includes directed light energy 106 to the arterial surface coupled with or without a photosensitizing agent. The light intensity can be modulated by an operator and/or a control unit to modulate light energy output over time in a dynamic manner, allowing increasing or decreasing intensity over time in a linear, logarithmic, exponential, step-wise or pulsatile manner.

In one embodiment, the light energy 106 is generated at the site of the light emitters 101a-101c. For example, the light energy 106 may be generated by light emitting diodes. The light energy may be actuated by an external user. The signal to turn the light energy on or off may be sent to the light emitters 101a-101c from a controller. In one example, the signal is sent to the light emitters 101a-101c through a wire extending the length of the catheter system 100.

In some embodiments, the catheter system 100 is used for imaging and photodynamic therapy in a human or animal patient. The patient may be given a photo-sensitizing agent in conjunction with the therapy. The photosensitizing agent is selectively taken up by specific cell types (e.g., inflammatory cell types), and when light energy 106 (photodynamic therapy) is aimed at a cell that has taken up the photosensitizing agent, the cell dies.

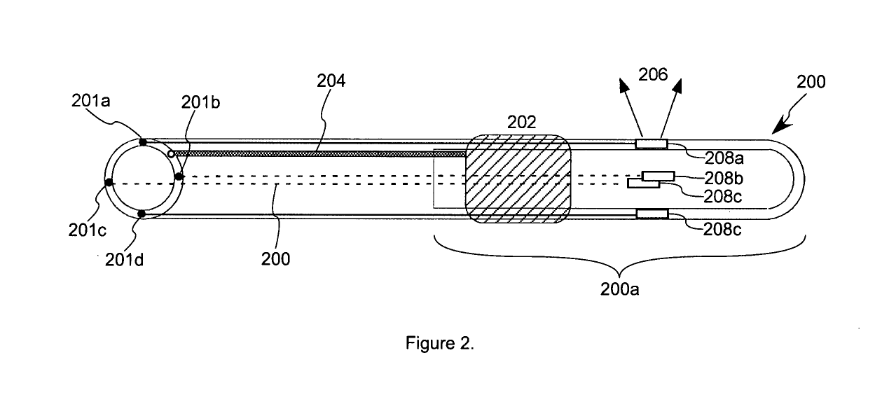

FIG. 2 depicts a partial cut-away view of a catheter system 200 with four light emitters 208a-208d and an ultrasound probe 202, according to an illustrative embodiment of the invention. The light emitter 208a emits light energy 206. The light energy 206 is generated by an external generator and transmitted through a fiber optic cable 201a. The fiber optic cable 201a extends from a first end of the catheter system (not shown) to the light emitter 208a. Similarly, fiber optic cables 201b-201d extend to the light emitters 208b-d. The light emitters 208a-208d are located at the distal end 200a of the catheter system 200.

The ultrasound imaging system 202 is a standard ultrasound probe connected to a control unit and imaging processing unit via independent cable 204. The light emitters 208a-208d include one or more light energy 206 outputs along the circumference of the catheter. The individual light emitters 208a-208d are positioned in an annular pattern to allow light distribution to the entire surrounding arterial surface. In one embodiment, light is transmitted only to a selected portion of the arterial wall through excitation of a selected portion of the light emitters 208a-208d. In this manner, unselected arterial surfaces are not exposed to the light energy 206.

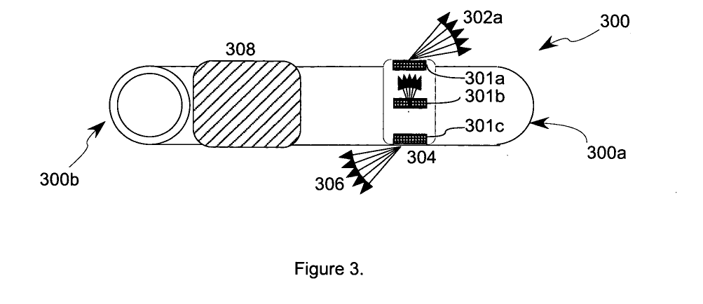

FIG. 3 depicts a side view of a catheter system 300, including an ultrasound imaging system 308 and light emitters 301a-301c emitting light energy at various angles 302a-302c, according to an illustrative embodiment of the invention. The catheter system 300 has a distal end 300a and a proximal end 300b. The light emitters 301a-301c emit light energy 302a-302c at various angles. Light emitter 301a emits light energy 302a toward a distal end 300a of the catheter system 300. Light emitter 301b emits light energy 302b perpendicular to the catheter system 300. Light emitter 301c emits light energy 302c toward a proximal end 300b of the catheter system 300. The light emitters 301a-301c may direct the light energy 302a-302c at various angles using any selected directing means. In one example, filters or prisms are used to direct the light energy 302a-302c. In another example, the light emitters 301a-301c are angled light sources. In another example, the light emitters 301a-301c are directed fiber optic light sources. The light source angle may be directed at a selected angle in order to target a selected layer or depth of tissue. For example, light may be directed to the deeper layers of a plaque from a light source distal or proximal to the plaque, without necessitating the entry of the light rays through the luminal layers above the plaque.

In various embodiments, the light emitters 301a-c are able to angle the light energy 302a-302c at angles ranging from about zero degrees to about 360 degrees around the x, y, and z-axes. In one embodiment, in which the z axis is perpendicular to the catheter, the light emitters are able to angle lights energy 302a-302c at angles ranging from about zero to about 360 degree around the x-axis and the y-axis, and at angles ranging from about zero to about 180 degrees on the z-axis.

In one example, a single round of plaque treatment in a patient may incorporate a series of light energy 302a-302c emissions, such that as the angle of light energy 302a-302c emitted changes with time, the intensity of light energy 302a-302c may also change. This method may be automated by a control unit or computer to allow automated catheter advancement or pull-back. Automation may enable even more specific dosing of a target area when coupled with changing angles and intensity of the light energy 302a-302c.

In various embodiments, the catheter system 300 may include multiple sites of light emitters 301a-301c along the length of the catheter system 300. In this embodiment, multiple atherosclerotic plaques may be stabilized simultaneously.

According to one aspect, a user controls the intensity, duration, and waveform of the light energy 302a-302c emitted from light emitters 301a-301c. In one example, the user is a doctor. The user uses images generated by use the ultrasound imaging system to identify target tissue (e.g, plaques or lesions), and through the use of these dynamic images, the user directs the light energy 302a-302c at the target tissue. The light energy 302a-302c may have a therapeutic effect on the target tissue. For example, it may reduce or remove inflammatory cells in atherosclerotic plaques.

It should be understood that in this specification, the words “light” and “light energy” are not limited to the visible part of the electromagnetic spectrum and includes parts of the electromagnetic spectrum outside the visible range of wavelengths.

Those skilled in the art will know or will be able to ascertain using no more than routine experimentation, many equivalents to the embodiments and practices described herein. Variations, modifications, and other implementations of what is described may be employed without departing from the spirit and scope of the invention. Any of the method, system and device features described above or incorporated by reference may be combined with any other suitable method, system or device features disclosed herein or incorporated by reference, and is within the scope of the contemplated inventions. The systems and methods may be embodied in other specific forms without departing from the spirit or essential characteristics thereof. The foregoing embodiments are therefore to be considered in all respects illustrative, rather than limiting of the invention. The teachings of all references cited herein are hereby incorporated by reference in their entirety. Accordingly, it will be understood that the invention is not to be limited to the embodiments disclosed herein, but is to be understood from the following claims, which are to be interpreted as broadly as allowed under the law.

We claim:

1. A catheter system for imaging and phototherapy, comprising:
a light emitter, and
an ultrasound imaging system coupled to the light emitter, wherein
the ultrasound imaging system transmits an image to a display device, and the light emitter emits light energy directed at a target location.
